(19) United States
(12) Patent Application Publication
(10) Pub. No.: US 2014/0094511 A1
(43) Pub. Date: Apr. 3, 2014

(54) Treatment of Obstructive Sleep Apnea Syndrome with a Combination of a Carbonic Anhydrase Inhibitor and an Additional Active Agent

(71) Applicant: Vivus, Inc., Mountain View, CA (US)

(72) Inventors: Leland F. Wilson, Menlo Park, CA (US); Peter Y. Tam, Redwood City, CA (US); Thomas Najarian, Los Osos, CA (US); Charles H. Bowden, Mountain View, CA (US)

(73) Assignee: Vivus, Inc., Mountain View, CA (US)

(21) Appl. No.: 14/056,109

(22) Filed: Oct. 17, 2013

Related U.S. Application Data
(63) Continuation of application No. 12/986,921, filed on Jan. 7, 2011, now abandoned.
(60) Provisional application No. 61/293,129, filed on Jan. 7, 2010.

(51) Int. Cl. A61K 31/357; A61K 31/165; A61K 31/137

(52) U.S. Cl. CPC A61K 31/357; A61K 31/137; A61K 31/165
USPC 514/454

(57) ABSTRACT

This invention relates generally to methods and pharmaceutical formulations useful in treating patients suffering from obstructive sleep apnea syndrome (OSAS). Treatment of OSAS is effected by administering a carbonic anhydrase inhibitor to the patient in combination with at least one additional active agent. Examples of additional active agents include modafinil, eszopiclone, zolpidem, zaleplon, and phentermine.

### RELATED APPLICATIONS

This application is a continuation of U.S. application Ser. No. 12/986,921, filed on Jan. 7, 2011, which claims priority from U. S. Provisional Application No. 61/293,129, filed on Jan. 7, 2010, each of which is incorporated by reference in its entirety.

### TECHNICAL FIELD

The invention relates generally to the treatment of sleep apnea with a carbonic anhydrase inhibitor, and more particularly relates to the treatment of obstructive sleep apnea syndrome with a combination of a carbonic anhydrase inhibitor and at least one additional active agent. The invention finds utility in the fields of medicine and pharmacotherapy.

### BACKGROUND

Apnea occurs when an individual breathes very shallowly or stops breathing completely over a time period of 10 seconds or more, resulting in a drop in blood oxygen level. Apneas usually occur during sleep and cause the individual to wake or transition from a deep level of sleep to a more shallow sleep state. “Hypopneas” refer to decreases in breathing that also result in hypoxemia, but are less severe than apneas. Generally, an apnea refers to a decrease in airflow or chest wall movement that is smaller than approximately 25% of baseline, while a hypopnea refers to a decrease of less than about 70% of baseline. See K. Banno et al. (2007) Sleep Medicine 8(4):400-426.

The International Classification of Sleep Disorders—2^{nd} edition (ICDS-2) defines two categories of sleep-related breathing disorders, central sleep apnea syndrome (CSAS) and obstructive sleep apnea syndrome (OSAS). Mixed sleep apneas involve both CSAS and OSAS. The distinction between CSAS and OSAS relates to the mechanism that causes the respiratory disturbance. CSAS involves a dysfunction in ventilatory control in the central nervous system (CNS), with a reduction in impulses transmitted from the CNS to the respiratory muscles. OSAS, which is much more common than CSAS, is a disorder that is caused by physical obstruction of the upper airway. The obstruction typically results from abnormal control of the muscles that maintain the patency of the upper airway, and/or abnormal craniofacial anatomy. Common risk factors for OSAS include obesity, enlarged tonsils and adenoids, and craniofacial abnormalities.

OSAS has emerged as a common sleep disorder that is associated with excessive daytime sleepiness as well as more significant problems, including atherosclerosis, hypertension, heart failure, nocturnal cardiac arrhythmias, and an elevated risk of myocardial infarction and stroke. See, e.g., Sleep Apnea: Implications in Cardiovascular and Cerebrovascular Disease, 2^{nd} Ed., Bradley et al., eds. (Informa Healthcare USA, Inc., 2010) (particularly Levitzky et al., Ch. 10, at p. 163; Friedman et al., Ch. 11, at p. 180; Lorenzo-Filho et al., Ch. 13, at p. 219; Siccoli et al., Ch. 14, at p. 237; Sorajja et al., ch. 15, at p. 261; and Yumino et al., ch. 17, at p. 302). A diagnosis of OSAS is typically made when repetitive apnea or hypopnea events occur during sleep, with 5-15 episodes/hour classified as mild OSAS, 15-30 episodes/hour classified as moderate OSAS, and over 30 episodes/hour classified as severe OSAS. Banno et al. (2007), citing Sleep-Related Breathing Disorders in Adults: Recommendations for Syndrome Definition and Measurement Techniques in Clinical Research, in Report of an American Academy of Sleep Medicine Task Force (1999), Sleep 22(5):667-689.

OSAS is commonly treated using the Continuous Positive Air Pressure (CPAP) technique, in which a continuous stream of compressed air is administered to the patient using a machine specifically designed for that purpose. Other forms of treatment include intraoral mandibular advancement devices and craniofacial surgery. These methods are cumbersome and expensive, and although many pharmacological agents have been proposed and evaluated, no agent has proved to be successful in treating OSAS.

There is, therefore, a need for a simpler, straightforward method for treating an individual with OSAS.

### SUMMARY OF THE INVENTION

The present invention addresses the aforementioned need in the art and provides a pharmacological treatment for patients suffering from OSAS. The treatment involves co-administration of a carbonic anhydrase inhibitor with an additional active agent or agents, such as modafinil and/or a sedative agent, preferably a nonbenzodiazepine sedative agent.

By “OSAS,” as the term is used herein, applicants are referring to obstructive sleep apnea syndrome as defined above, but do not intend to exclude the possibility that the individual being treated may also have some degree of CSAS.

In addition, by “treating OSAS” applicants are referring to (1) the elimination of nighttime apneas and/or hypopneas, (2) a reduction in the number of apneas and/or hypopneas per hour and/or per night, and/or (3) the amelioration of the extent of each apnea and/or hypopnea event experienced by the individual undergoing treatment (as may be determined, for instance, by an increase in airflow or in the amplitude of chest wall movement). While some methods of treatment and pharmaceutical formulations herein may also alleviate excessive daytime sleepiness, particularly when treatment involves administration of modafinil and/or a sympathomimetic amine such as phentermine, any such effect is incidental to the present method, which treats OSAS by virtue of effecting (1), (2), or (3) as explained above.

In one aspect, then, the invention provides a method for treating OSAS in a patient by co-administering to the patient:

(a) a therapeutically effective amount of a carbonic anhydrase inhibitor; and

(b) a therapeutically effective amount of an additional active agent selected from modafinil, a nonbenzodiazepine sedative agent, and combinations thereof. The modafinil can be in the form of a racemic mixture of its two enantiomers, or it can be an isolated enantiomer (i.e., the R-enantiomer or the S-enantiomer, as will be explained in detail infra). Modafinil may also be in a crystalline form, and/or in the form of a prodrug, a conjugate, an active metabolite, or present as another such derivative, analog, or related compound known to those of ordinary skill in the art or that may be discovered.

In another aspect, the invention provides a method for treating OSAS in a patient by co-administering to the patient:

(a) a therapeutically effective amount of a carbonic anhydrase inhibitor; and

(b) a therapeutically effective amount of modafinil.

In another aspect, the invention provides a method for treating OSAS in a patient by co-administering to the patient:

(a) a therapeutically effective amount of a carbonic anhydrase inhibitor;

(b) a therapeutically effective amount of modafinil as above; and

(c) a therapeutically effective amount of a nonbenzodiazepine sedative agent as noted above, e.g., zopiclone, eszopiclone, zolpidem, zaleplon, gaboxadol, indiplon, or the like.

In a further aspect, the invention provides a method for treating OSAS in a patient by co-administering to the patient:

(a) a therapeutically effective amount of a carbonic anhydrase inhibitor; and

(b) a therapeutically effective amount of a nonbenzodiazepine sedative agent as noted above.

In a further aspect, the invention provides a method for treating OSAS in a patient by co-administering to the patient:

(a) a therapeutically effective amount of a carbonic anhydrase inhibitor;

(b) a therapeutically effective amount of modafinil as above; and

(c) a therapeutically effective amount of a sympathomimetic amine, where the sympathomimetic amine may be, for example, phentermine, bupropion, chlorphentermine, or the like.

Any inhibitor of carbonic anhydrase can be used in the present methods and formulations. Without wishing to be bound by theory, applicants postulate that the effectiveness of including a carbonic anhydrase inhibitor in a combination therapy to treat OSAS results from the body's response to the metabolic acidosis caused by the drug (in contrast to the respiratory acidosis often experienced by OSAS sufferers). That is, as inhibition of carbonic anhydrase results in a decrease in blood pH (the reaction catalyzed by carbonic anhydrase is the reversible hydrolysis of carbon dioxide to give bicarbonate ion and a proton), the body works to compensate by breathing faster and deeper to expel excess carbon dioxide and thus restore equilibrium. Topiramate, zonisamide, and acetozolamide are representative of the many carbonic anhydrase inhibitors that may be used in the context of the present invention.

In another aspect of the invention, a method is provided for treating obstructive sleep apnea syndrome in a patient, comprising orally administering to the patient: a therapeutically effective amount of a carbonic anhydrase inhibitor selected from acetazolamide, brinzolamide, diclofenamide, dichlorphenamide, dorzolamide, furosemide, imidazole, methazolamide, phenylalanine, topiramate, and zonisamide; and a therapeutically effective amount of modafinil. In a variation, the method further includes co-administration of a therapeutically effective amount of phentermine.

In another aspect of the invention, a method is provided for treating obstructive sleep apnea in a patient, comprising orally administering to the patient, on a daily basis, a therapeutically effective amount of topiramate and a therapeutically effective amount of phentermine.

In a further aspect of the invention, a method is provided for treating obstructive sleep apnea in a patient, comprising orally administering to the patient, on a daily basis, a therapeutically effective amount of topiramate, a therapeutically effective amount of phentermine, and a therapeutically effective amount of modafinil.

The invention additionally pertains to pharmaceutical formulations useful in the present methods of treating OSAS:

In one aspect of this embodiment, a pharmaceutical formulation for the treatment of OSAS is provided, the formulation comprising a therapeutically effective amount of a carbonic anhydrase inhibitor, a therapeutically effective amount of a sympathomimetic amine, and a therapeutically effective amount of modafinil.

In one aspect of this embodiment, a pharmaceutical formulation for the treatment of OSAS is provided, the formulation comprising a therapeutically effective amount of a carbonic anhydrase inhibitor, a therapeutically effective amount of a nonbenzodiazepine sedative agent, and a therapeutically effective amount of modafinil.

In another aspect of this embodiment, a pharmaceutical formulation for the treatment of OSAS is provided, the formulation comprising a therapeutically effective amount of a sulfonamide carbonic anhydrase inhibitor and a therapeutically effective amount of modafinil.

In another aspect of this embodiment, a pharmaceutical formulation for the treatment of OSAS is provided, the formulation comprising a therapeutically effective amount of a sulfonamide carbonic anhydrase inhibitor and a therapeutically effective amount of a nonbenzodiazepine sedative agent.

In a further aspect of this embodiment, a pharmaceutical formulation is provided that comprises topiramate, phentermine, and modafinil.

In a further aspect of this embodiment, a pharmaceutical formulation is provided that comprises topiramate, bupropion, and modafinil.

In a further aspect of this embodiment, a pharmaceutical formulation is provided that comprises zonisamide, phentermine, and modafinil.

In a further aspect of this embodiment, a pharmaceutical formulation is provided that comprises zonisamide, bupropion, and modafinil.

In a further aspect of this embodiment, a pharmaceutical formulation is provided that comprises zonisamide and modafinil.

In another embodiment, the invention provides a packaged pharmaceutical preparation comprising:

a carbonic anhydrase inhibitor and (a) modafinil, (b) a nonbenzodiazepine sedative agent, (c) both modafinil and a nonbenzodiazepine sedative agent, or (d) modafinil and a sympathomimetic amine; and

instructions for administering, e.g., self-administering, the active agents in the treatment of OSAS. The active agents are present in amounts that are therapeutically effective for the treatment of OSAS, and may be in separate dosage forms or combined in a single dosage form, where the dosage forms are usually but not always orally administrable. The instructions for administration may include reference to an escalating dosing regimen wherein a lower daily dosage of one or more active agents is administered initially, with incremental increases at various designated time points thereafter. Ideally, a titration card is provided that sets forth the recommended dosages for at least four weeks.

### DETAILED DESCRIPTION OF THE INVENTION

It must be noted that, as used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the invention pertains. Specific terminology of particular importance to the description of the present invention is defined below. Unless defined otherwise, all technical and scientific terms used herein have the meaning commonly understood by one of ordinary skill in the art to which the invention pertains.

When referring to an active agent, applicants intend the term “active agent” to encompass not only the specified molecular entity but also its pharmaceutically acceptable, pharmacologically active analogs, including, but not limited to, salts, esters, amides, prodrugs, conjugates, active metabolites, crystalline forms (including polymorphs), enantiomers, and other such derivatives, analogs, and related compounds.

By the terms “effective amount” and “therapeutically effective amount” of a compound is meant a nontoxic but sufficient amount of an active agent to provide the desired effect, i.e., treatment of OSAS as manifested by the elimination of nighttime apneas and/or hypopneas, a reduction in the number of apneas and/or hypopneas per hour and/or per night, and/or amelioration of the extent of each apnea and/or hypopnea event experienced by the individual undergoing treatment.

The term “unit dosage form” denotes any form of a pharmaceutical formulation that contains an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient provides a measurable time delay before drug is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

The invention involves administration of a carbonic anhydrase inhibitor and at least one additional active agent to a patient suffering from OSAS. Carbonic anhydrase inhibitors are generally imidazoles (such as imidazole per se), imidazole derivatives, sulfamates (such as topiramate), and sulfonamides (such as zonisamide). Any carbonic anhydrase inhibitor may be advantageously employed in conjunction with the present invention. Examples of suitable carbonic anhydrase inhibitors include, without limitation, acetazolamide (Diamox™), brinzolamide, diclofenamide, dichlorphenamide (Daranide™), dorzolamide, furosemide, imidazole, methazolamide (Neptazane™), phenylalanine, topiramate, and zonisamide. Carbonic anhydrase inhibitors also include selective inhibitors of the cyclooxygenase-2 enzyme (“cox 2 inhibitors”), such as such as celecoxib, valdecoxib, rofecoxib, etoricoxib, and the like. Preferred carbonic anhydrase inhibitors for use in conjunction with the present invention include, without limitation, acetazolamide, brinzolamide, diclofenamide, dichlorphenamide, dorzolamide, furosemide, imidazole, methazolamide, phenylalanine, topiramate, zonisamide, celecoxib, valdecoxib, rofecoxib, and etoricoxib, with acetazolamide, zonisamide, and topiramate particularly preferred. The daily dose of topiramate effective to treat OSAS according to the method of the invention, when administered orally, is generally in the range of about 5 mg to about 800 mg, more typically in the range of about 5 mg to about 400 mg, preferably in the range of about 25 mg to about 250 mg, and optimally in the range of about 25 mg to about 100 mg. The daily dose may be undivided, such that carbonic anhydrase inhibitor is administered once a day, or the daily dose may be divided into two to four individual doses. Preferably, the topiramate is administered in sustained release form, as will be discussed infra, either once or twice daily to achieve a daily dosage in the aforementioned ranges. It will be appreciated that the daily dose of topiramate as well as other carbonic anhydrase inhibitors normally represents on the order of 25% to 200%, more generally 25% to 100%, and most typically 25% to 75%, of the daily dose known and/or prescribed for previously known indication(s) (as set forth, for example, in the Physicians' Desk Reference), using the same mode of administration.

In a preferred embodiment, the dosage of the carbonic anhydrase inhibitor is increased gradually at the outset of therapy, generally over a period of about three to ten weeks, more usually over a period of about three to about eight weeks, starting with a relatively low initial dose, in order to reduce the likelihood of undesirable side effects. With topiramate, for example, a representative dosage regimen is as follows: administration of about 25 mg daily for about the first 5-7 days of treatment; administration of about 50 mg daily for the next 5-7 days; administration of about 75 mg daily for about the next 5-7 days; administration of about 100 mg daily for the next 5-7 days; and, subsequently, ongoing administration of a daily maintenance dose in the ranges specified earlier herein.

In one embodiment, the additional active agent is modafinil. Modafinil is an agent with activity in the central nervous system and was developed as a treatment for the excessive daytime sleepiness associated with narcolepsy. The primary pharmacological activity of modafinil, like amphetamine-like agents, is to promote wakefulness, initially in the treatment of patients with narcolepsy, as noted, and more recently in the reduction of daytime sleepiness associated with OSAS and sleep work shift disorder (SWSD). Aside from reducing the daytime sleepiness associated with OSAS, modafinil has not been approved for treatment of OSAS per se, nor has its utility in the treatment of obstructive sleep apnea per se been recognized.

Modafinil, also known as 2-[(diphenylmethypsulfinyl]-N-acetamide or benzhydrylsulphinyl acetamide, is a synthetic acetamide derivative, the structure and synthesis of which are described in U.S. Pat. No. 4,177,290 to Lafon. Modafinil's formula is C₁₅H₁₅NO₂S and its molecular weight is 273.35 g/mol. Modafinil is insoluble in water and cyclohexane, sparingly or slightly soluble in methanol and acetone. The racemic compound has a melting point of 163-165° C. Modafinil has an asymmetric center at the sulfur atom and thus exists as two optical isomers, i.e., enantiomers. Synthesis of the individual enantiomers has been described; see, e.g., U.S. Pat. No. 7,317,126 to Rebiere et al., and the R-enantiomer (also referred to as “armodafinil” has shown therapeutic utility when administered individually. Various analogs and derivatives of modafinil have also been described, including various crystalline and polymorphic forms; see U.S. Pat. Nos. 6,992,219 to Broquaire et al. and 7,132,570 to Neckebrock. All of these forms, analogs, derivatives, enantiomers, etc., as noted earlier herein, are intended to be encompassed by the term “modafinil.”

Modafinil is available commercially as Provigil®, manufactured and marketed by Cephalon, Inc. of West Chester, Pa. Provigil® is supplied as tablets containing 100 mg or 200 mg modafinil. Accordingly, therapeutic packages providing one or more unit doses of modafinil as an active ingredient thereof are commercially available in a finished pharmaceutical container. In the provided literature accompanying a pharmaceutical container are instructions that the daily dosage of modafinil for treating daytime sleepiness is 200 mg/day given as a single dose in the morning. As with carbonic anhydrase inhibitors such as topiramate, the preferred oral daily dosage of modafinil herein, i.e., in the context of the present method, is on the order of 25% to 200%, more generally 25% to 100%, and most typically 25% to 75%, of the daily dose known and/or prescribed for previously known indication(s). Accordingly, a preferred daily dose of modafinil in the present context is in the range of about 50 mg to 400 mg, more generally about 50 mg to 200 mg, and most typically about 50 mg to about 150 mg. With administration of armodafinil, i.e., the R-enantiomer of modafinil in enantiomerically pure or enriched form, preferred daily doses will be approximately half of those specified above.

In another embodiment, the additional active agent that is co-administered with the carbonic anhydrase inhibitor, e.g., topiramate, is a nonbenzodiazepine sedative agent. Examples of nonbenzodiazepine sedative agents for use herein include, without limitation, zopiclone, eszopiclone, zolpidem, zaleplon, gaboxadol, and indiplon, with preferred oral daily dosages herein corresponding to 25% to 200%, more generally 25% to 100%, and most typically 25% to 75%, of the known and/or prescribed daily doses for these active agents.

In a further embodiment, the additional active agent comprises a mixture of at least two active agents. In one example of such a case, the carbonic anhydrase inhibitor is administered with both modafinil and a nonbenzodiazepine sedative agent as described above.

In still another embodiment of the invention, a method for treating OSAS is provided that comprises administering a therapeutically effective amount of a carbonic anhydrase inhibitor as described above in combination with a therapeutically effective amount of modafinil, as also described above, wherein the method further includes co-administering a therapeutically effective amount of a sympathomimetic amine.

Sympathomimetic amines, including the catecholamines, are amine drugs that mimic the actions of drugs that activate the sympathetic nervous system, such as epinephrine and norepinephrine. Sympathomimetic amines thus include amphetamine, benzphetamine, bupropion, chlorphentermine, colterol, diethylpropion, dopamine, dobutamine, ephedrine, epinephrine, epinine, ethylnorepinephrine, fenfluramine, fenoldapam, hydroxyamphetamine, ibopamine, isoetharine, isoproterenol, mephentermine, metaproterenol, metaraminol, methoxamine, methoxyphenamine, midodrine, norepinephrine, phendimetrazine, phenmetrazine, phentermine, phenylephrine, phenylethylamine, phenylpropanolamine, prenalterol, propylhexedrine, protokylol, ritodrine, terbutaline, tuaminoheptane, tyramine, and acid addition salts thereof, either organic or inorganic. Common acid addition salts of some of the aforementioned sympathomimetic amines include, without limitation, dobutamine hydrochloride, epinephrine bitartrate, ethylnorepinephrine hydrochloride, fenoldopam mesylate, hydroxyamphetamine hydrobromide, isoproterenol hydrochloride, mephentermine sulfate, metaraminol bitartrate, methoxamine hydrochloride, norepinephrine bitartrate, phenylephrine hydrochloride, and terbutaline sulfate.

Preferably, the sympathomimetic amine is phentermine, bupropion, or chlorphentermine, with phentermine and bupropion particularly preferred. In an exemplary embodiment, the carbonic anhydrase inhibitor administered is topiramate and the sympathomimetic amine administered is phentermine, wherein the daily dose of topiramate is as given above and the corresponding daily dose of phentermine that is co-administered is such that the weight ratio of the daily dose of topiramate to the daily dose of phentermine is in the range of about 2.5:1 to about 20:1, typically in the range of about 5:1 to about 20:1. In another exemplary embodiment, the carbonic anhydrase inhibitor administered is topiramate and the sympathomimetic amine administered is bupropion, wherein preferred daily doses of topiramate are as given above, and the corresponding daily dose of bupropion that is co-administered is such that the weight ratio of the daily dose of topiramate to the daily dose of bupropion is in the range of about 1:5 to about 3:1, preferably in the range of about 1:4 to about 2:1, most preferably in the range of about 1:4 to about 1.5:1

Administration of the active agents may be carried out using any appropriate mode of administration. Thus, administration can be, for example oral or parenteral, although oral administration is preferred.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical formulations and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). Oral administration and therefore oral dosage forms are generally preferred, and include tablets, capsules, caplets, solutions, suspensions and syrups, and may also comprise a plurality of granules, beads, powders, or pellets that may or may not be encapsulated. Preferred oral dosage forms are capsules and tablets.

As noted above, it is especially advantageous to formulate compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of each active agent, such that the unit dosage in each tablet or capsule is approximately 50% of the therapeutically effective amount.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are also preferred oral dosage forms, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited earlier herein, which describes materials and methods for preparing encapsulated pharmaceuticals.

Oral dosage forms, whether tablets, capsules, caplets, or particulates, may, if desired, be formulated so as to provide for controlled release of the carbonic anhydrase inhibitor and/or the additional active agent(s), and in a preferred embodiment, the present formulations are controlled release oral dosage forms. Generally, the dosage forms provide for sustained release, i.e., gradual, release of one or more of the active agent(s), particularly the carbonic anhydrase inhibitor, from the dosage form to the patient's body over an extended time period, typically providing for a substantially constant blood level of the agent over a time period in the range of about 4 to about 12 hours, typically in the range of about 6 to about 10 hours. In a particularly preferred embodiment, there is a very gradual increase in blood level of the drug following oral administration of the dosage form containing the carbonic anhydrase inhibitor, such that peak blood level (generally about 50-200 μg/ml for topiramate, about 1-5 μg/ml for zonisamide, or about 10-35 pg/ml for acetazolamide), is not reached until at least 4-6 hours have elapsed, with the rate of increase of blood level drug approximately linear. In addition, in the preferred embodiment, there is an equally gradual decrease in blood level at the end of the sustained release period.

Generally, as will be appreciated by those of ordinary skill in the art, sustained release dosage forms are formulated by dispersing the active agents within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preferred sustained release dosage forms herein are composed of the acrylate and methacrylate copolymers available under the tradename “Eudragit” from Rohm Pharma (Germany). The Eudragit series E, L, S, RL, RS, and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The active agent may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the active agents may be formulated in a depot preparation for controlled release of the active agents, preferably sustained release over an extended time period. These sustained release dosage forms are generally administered by implantation (e.g., subcutaneously or intramuscularly or by intramuscular injection).

In combining the active agents herein, i.e., the carbonic anhydrase inhibitor with (1) modafinil, (2) modafinil and a nonbenzodiazepine sedative, (3) a nonbenzodiazepine sedative, or (4) modafinil and a sympathomimetic amine such as phentermine, the additional active agents will in some cases reduce the quantity of the carbonic anhydrase inhibitor needed to achieve a therapeutic effect, e.g., a sympathomimetic amine such as phentermine or bupropion can reduce the minimum effective amount of a carbonic anhydrase inhibitor such as topiramate, zonisamide, or acetazolamide.

As the method of the invention involves combination therapy, the active agents may be administered separately, at the same or at different times of day, or they be administered in a single pharmaceutical formulation. In the embodiment wherein a sympathomimetic amine is administered with modafinil and with a carbonic anhydrase inhibitor such as topiramate, it is generally preferred that the sympathomimetic amine be administered earlier in the day than the carbonic anhydrase inhibitor. In an analogous method, the dosage form can contain the carbonic anhydrase inhibitor as well as the modafinil and the sympathomimetic amine, with the sympathomimetic amine preferably in immediate release form and the carbonic anhydrase inhibitor and the modafinil optionally in controlled release form. As an example, a combination dosage form of the invention for once-daily administration might contain (1) modafinil in the therapeutically effective amounts specified earlier herein, (2) in the range of about 5 mg to about 800 mg topiramate, preferably about 25 mg to about 250 mg topiramate, and optimally about 25 mg to about 100 mg topiramate, in controlled release (e.g., sustained release) form, and (3) either phentermine in immediate release form, or bupropion in controlled release form, with the additional active agent present in an amount that provides a weight ratio of topiramate to phentermine, or a weight ratio of topiramate to bupropion, specified as above. In other formulations of the invention, two or more additional active agents, which may or may not be in the same class of drug (e.g., sympathomimetic amines), can be present in combination, along with the carbonic anhydrase inhibitor. In such a case, the effective amount of either or each individual additional active agent present will generally be reduced relative to the amount that would be required if only a single added agent were used. Specific examples of such once-daily formulations include the following:

(1) 200 mg topiramate, 15 mg phentermine; 50 mg modafinil;

(2) 200 mg topiramate, 10 mg phentermine; 200 mg modafinil;

(3) 150 mg topiramate, 15 mg phentermine; 50 mg modafinil;

(4) 150 mg topiramate, 10 mg phentermine; 100 mg modafinil;

(5) 100 mg topiramate, 15 mg phentermine; 50 mg modafinil;

(6) 100 mg topiramate, 10 mg phentermine; 200 mg modafinil;

(7) 200 mg topiramate, 300 mg bupropion; 100 mg modafinil;

(8) 200 mg topiramate, 250 mg bupropion; 200 mg modafinil;

(9) 200 mg topiramate, 200 mg bupropion; 100 mg modafinil;

(10) 200 mg topiramate, 150 mg bupropion; 50 mg modafinil;

(11) 200 mg topiramate, 100 mg bupropion; 400 mg modafinil;

(12) 100 mg topiramate, 300 mg bupropion; 200 mg modafinil;

(13) 100 mg topiramate, 250 mg bupropion; 100 mg modafinil;

(14) 100 mg topiramate, 200 mg bupropion; 50 mg modafinil;

(15) 100 mg topiramate, 150 mg bupropion; 200 mg modafinil; and

(16) 100 mg topiramate, 100 mg bupropion; 100 mg modafinil.

As may be deduced from the foregoing, representative topiramate/phentermine formulations typically contain, in addition to 50 mg, 100 mg, 150 mg, or 200 mg modafinil, 100 mg to 200 mg topiramate and: 100 mg to 300 mg bupropion; 10 mg to 15 mg phentermine; or 100 mg to 300 mg bupropion and 5 mg to 10 mg phentermine.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description is intended to illustrate and not limit the scope of the invention. I t will be understood by those skilled in the art that various changes may be made and equivalents may be substituted without departing from the scope of the invention, and further that other aspects, advantages and modifications will be apparent to those skilled in the art to which the invention pertains.

Formulations containing a carbonic anhydrase inhibitor and at least one additional active agent can be prepared as described herein and/or as described in U.S. Pat. Nos. 7,056,890 and 7,533,818 to Najarian, and in U.S. Patent Publication Nos. 2008/0255093 to Tam et al., 2008/0312163 to Najarian et al., all of common assignment herewith to Vivus, Inc. (Mountain View, Calif.). Evaluation of the combination therapy of the present invention in the treatment of OSAS can be carried out using known in vitro and in vivo techniques as described, for example, in See, e.g., Sleep Apnea: Implications in Cardiovascular and Cerebrovascular Disease, 2^{nd} Ed., Bradley et al., eds. (Informa Healthcare USA, Inc., 2010).

### EXAMPLE

Various techniques known to those in the art and/or described in the pertinent literature and texts can be implemented to demonstrate the efficacy of the present combinations in the treatment of OSA.

Evaluation of Wake Promoting Activity in Rats: The methodology described by Edgar et al. (1997) J. Pharmacol. Exper. Therap. 283:757-769 can be carried out to evaluate the wake promoting activity of the inventive compositions. Specifically, male Wistar rats are anesthetized and surgically prepared with implants for recording chronic EEG (encephalographic) and EMG (electromyographic) activity. During the week following surgical introduction of the implants, the rats are kept in a controlled environment with antibiotics administered to prevent infection. After the week allowed for post-surgical recovery, the compositions are evaluated on groups of from 4 to 8 rats carried out over one or two separate test session. Each animal is tested with a different composition for up to ten weeks with at least seven days between successive tests. A control group is included in each experiment, with the control group receiving modafinil and/or a nonbenzodiazepine sedative, but no carbonic anhydrase inhibitor or sympathomimetic amine. Dosing is carried out at the same time each day using a selected mode of administration, e.g., intraperitoneal injection in a volume of approximately 5 mL/kg. Sleep/wake scoring is carried out by manually determining sleep and wake activity using available software, e.g., Icelus software developed by Mark Opp at the University of Michigan, Ann Arbor. EEG and EMG signals are detected using commercially available or otherwise known means; the Icelus program displays the detected EEG and EMG values in blocks of six seconds. Arousal state is scored as awake, rapid eye movement (REM), or slow-wave or non-REM sleep (NREM), according to visual analysis of EEG frequency and amplitude characteristics and EMG activity, as is now known in the art (see Opp et al. (1994) Amer. J. Physiol. 266:R688-95; van Gelder (1991) Sleep 14:48-55; Edgar et al. (1997) J. Pharmacol. Exp. Ther. 283:757-69, and the like). Two factors can be used to ascertain whether a tested composition exhibits wake-enhancing activity. The first is the relative amount of awake time during the thirty-minute period following dosing; the second is the total awake time in the first three hours following dosing). All activity values are compared against the corresponding control value(s) in each test. Compositions of the invention are expected to exhibit far greater awake time using either mode of evaluation, thus demonstrating utility for wake-promoting activity in the aforementioned tests. The compositions of the invention are also expected to demonstrate utility in wake-promoting activity using alternative tests which are known or have yet to be developed.

Evaluation in Human Patients Suffering from OSA: Patients suffering from OSA participate in a sleep study in which air flow and pressure are monitored throughout the night, during both inspiration and expiration. Air flow and pressure are measured using a pneumotacograph, which is contained in a mask covering the patient's nose and mouth and works by measuring a pressure drop across a linear resistance. Prior to evaluation of the compositions of the invention using this method, each patient's apnea is analyzed with respect to the degree, frequency, and timing of changes in air flow and pressure. When measured air flow is zero, the apnea is occurring; this is normally in the middle or end of expiration. The frequency of apnea events gives rise to the apnea index (AI), defined as the number of apnea events per unit of time, usually the number of apnea events per hour. The compositions of the invention are administered to patients to evaluate the reduction in the severity of apnea events (as measured by a relative increase in air flow) as well as the AI.

1. A method for treating obstructive sleep apnea syndrome in a patient, comprising co-administering to the patient a therapeutically effective amount of a carbonic anhydrase inhibitor and a therapeutically effective amount of an additional active agent selected from modafinil, a nonbenzodiazepine sedative agent, and combinations thereof.

2.-28. (canceled)
